(19) United States
(12) Patent Application Publication
(10) Pub. No.: US 2024/0197816 A1
(43) Pub. Date: Jun. 20, 2024

(54) COMPOSITION AND METHOD FOR PROLONG SURVIVAL OF TRANSPLANT AND RECIPIENT

(71) Applicants: NovMetaPharma Co., Ltd., Seoul (KR); Seoul National University R&DB Foundation, Seoul (KR); SEOUL NATIONAL UNIVERSITY HOSPITAL, Seoul (KR)

(72) Inventors: Seung Hee Yang, Seoul (KR); Hoeyune Jung, Pohang-si (KR); Jong Min Kim, Seoul (KR); Yon Su Kim, Seoul (KR); Heonjong Lee, Incheon (KR)

(73) Assignees: NovMetaPharma Co., Ltd., Seoul (KR); Seoul National University R&DB Foundation, Seoul (KR); SEOUL NATIONAL UNIVERSITY HOSPITAL, Seoul (KR)

(21) Appl. No.: 18/533,699

(22) Filed: Dec. 8, 2023

Related U.S. Application Data
(60) Provisional application No. 63/386,531, filed on Dec. 8, 2022.

(51) Int. Cl. A61K 38/12; A61K 45/06; A61P 37/06

(52) U.S. Cl. CPC A61K 38/12; A61K 45/06; A61P 37/06

(57) ABSTRACT

A treatment of rejection of a transplant by a recipient of the transplant is disclosed. Methods for prolonging transplant survival in a recipient of the transplant, prolonging survival of the recipient, delaying and/or suppressing delayed graft function in the recipient, and/or reducing the amount of an immunosuppressant administered for transplantation. The methods include providing the transplant with a CHP or a pharmaceutically acceptable salt thereof is disclosed. Also provided is a method for the production of a pharmaceutical composition for the treatment of a transplant allowing modulating transplant survival in a recipient of the transplant.

### CROSS REFERENCE TO RELATED APPLICATIONS

This application claims priority to and benefit of U.S. Provisional Application No. 63/386,531 filed Dec. 8, 2022, of which the entire content is incorporated by reference.

### TECHNICAL FIELD

The present disclosure relates to a treatment of a transplant in an animal recipient for prolonging survival of the transplant in the recipient. The present disclosure relates to prolonging survival of an allograft in a recipient by administering a CHP or a pharmaceutically acceptable salt thereof to the recipient. Also the present disclosure is directed to a method are useful in preventing graft rejection and/or decreasing the amount of an immunosuppressant used at the time of transplant and/or for a maintenance after the transplant.

### BACKGROUND

Organ transplantation is the preferred treatment for most patients with chronic organ failure. Although transplantation of kidney, liver, lung, and heart offers an excellent opportunity for rehabilitation as recipients return to a more normal lifestyle, it is limited by the medical/surgical suitability of potential recipients, an increasing shortage of donors, and premature failure of transplanted organ function.

Despite great improvement in treatments to inhibit graft rejection (or transplant rejection), rejection continues to be the single largest impediment to successful organ transplantation. Rejection includes not only acute rejection but also chronic rejection. According to OPTN/SRTR Annual Report, 2022, 6.8% of adult kidney transplant recipients in 2018-2019 experienced acute rejection by 1 year, including 9.1% of recipients aged 18-34 and 5.9% of recipients with age of 65 years or greater. Acute rejection at 1 year occurred in 8.4% of those who received interleukin-2 (IL-2)-receptor antibody induction, compared with 6.6% who received T-cell depleting induction and 6.4% of the small subgroup whose transplants were managed without induction. For the livers, one-year survival rates for transplanted liver is about 90%, and the five year graft survival rate is about 75%, and 11.5% of adult liver transplant recipients in 2018-2019 reported at least one episode of acute rejection within 1 year. See OPTN/SRTR Annual Report, 2022.

In order to inhibit the detrimental immune reactions during transplantation and first a few month after the transplantation (induction phase) as well as post-transplantation long term (maintenance phase, immunosuppressive drugs (such as cyclosporin A, tacrolimus, and corticosteroids) or antibody therapies (such as anti-T cell antibodies) are generally administered. Unfortunately, the immunosuppression generally has undesirable side effects. For example, cyclosporin may cause decreased renal function, hypertension, toxicity and it must be administered for the life of the patient. Corticosteroids may cause decreased resistance to infection, painful arthritis, osteoporosis and cataracts. The anti-T cell antibodies may cause fever, hypertension, diarrhea or sterile meningitis and are quite expensive.

Accordingly, there have been needs for developing methods or therapies that induce unresponsiveness or tolerance in the host to a transplant, and/or to prolong survival of a graft, and/or a recipient of the transplant, and/or prevent or delay the graft loss, and/or suppress delayed graft function, and/or reducing the amount of an immunosuppressant used during and/or after the transplantation.

### SUMMARY

According to an aspect of the present disclosure, a method of suppressing an immune response comprising administering an effective amount of a cyclo histidine-proline (cyclo His-Pro or CHP), a pharmaceutically acceptable salt thereof, a stereoisomer, or a solvate thereof to an animal in need of such treatment. In the embodiment, the immune response may include acute rejection and/or chronic rejection of transplant by an animal recipient. According to another aspect, the method may further comprise administering an immunosuppressant. In an embodiment, the immune response may be a transplant rejection. The CHP, a pharmaceutically acceptable salt thereof, a stereoisomer, or a solvate thereof, optionally with an immunosuppressant may be administered during a transplantation operation, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

In another embodiment, the present disclosure provides a method of suppressing an immune response to a transplanted organ, tissue or cell, comprising administering an effective amount of a CHP, a pharmaceutically acceptable salt thereof, a stereoisomer, or a solvate thereof to a mammal in need thereof. In the embodiment, the transplanted organ, tissue or cell includes organs, tissues, or cells of lung, liver, kidney, heart, pancreas, intestine, abdominal wall, face/scalp, uterus, penis, and the like. In the embodiment, the immune response may include acute rejection and/or chronic rejection of transplant by an animal recipient. According to another aspect, the method may further comprise administering an immunosuppressant. The CHP, a pharmaceutically acceptable salt thereof, a stereoisomer, or a solvate thereof, and the immunosuppressant may be administered during a transplantation operation, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

Still another aspect of the present disclosure provides a method of prolong a survival of graft, delaying or preventing a graft loss, and/or suppressing delayed graft function in a recipient animal comprising treating the transplant with a composition comprising a CHP, a pharmaceutically acceptable salt thereof, a stereoisomer, or a solvate thereof. In the embodiment, the transplant includes organs, tissues, or cells of lung, liver, kidney, heart, pancreas, intestine, abdominal wall, face/scalp, uterus, penis, and the like. According to another aspect, the method may further comprise administering an immunosuppressant. When used as a combination, the CHP, a pharmaceutically acceptable salt thereof, a stereoisomer, or a solvate thereof, and optionally the immunosuppressant may be administered during a transplantation operation, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

Yet still another aspect of the present disclosure provides a method of prolong a survival of an animal recipient of transplant comprising administering to the animal recipient an effective amount of a composition comprising a CHP, a pharmaceutically acceptable salt thereof, a stereoisomer, or a solvate thereof. In the embodiment, the transplant includes organs, tissues, or cells of lung, liver, kidney, heart, pancreas, intestine, abdominal wall, face/scalp, uterus, penis, and the like. According to another aspect, the method may further comprise administering an immunosuppressant. When used as a combination, the CHP, a pharmaceutically acceptable salt thereof, a stereoisomer, or a solvate thereof may be administered during a transplantation operation, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

Another aspect of the present disclosure provides a method of prolong a survival of a graft, delaying or preventing a graft loss, and/or suppressing delayed graft function in an animal recipient administering to the animal recipient an effective amount of a composition comprising a CHP, a pharmaceutically acceptable salt thereof, a stereoisomer, or a solvate thereof is disclosed. In the embodiment, the transplant includes organs, tissues, or cells of lung, liver, kidney, heart, pancreas, intestine, abdominal wall, face/scalp, uterus, penis, and the like. According to another aspect, the method may further comprise administering an immunosuppressant. When used as a combination, the CHP, a pharmaceutically acceptable salt thereof, a stereoisomer, or a solvate thereof may be administered during a transplantation operation, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

An aspect of the present disclosure also includes compositions containing a CHP, a pharmaceutically acceptable salt thereof, a stereoisomer, or a solvate thereof for use in suppressing an immune response. The composition may further comprise an immunosuppressant, or may be administered in combination with an immunosuppressant. The composition, optionally in combination with an immunosuppressant may be administered during a transplantation operation (induction phase) and/or after the transplantation operation for the long term (maintenance phase). When used as a combination, the composition and the immunosuppressant may be administered simultaneously or sequentially, and may be formulated into a single formulation or different formulations.

An aspect of the present disclosure also includes compositions containing a CHP, a pharmaceutically acceptable salt thereof, a stereoisomer, or a solvate thereof for use in prolonging survival of a transplant in an animal recipient and/or in prolonging survival of an animal recipient of a transplant. In the embodiment, the transplant includes organs, tissues, or cells of lung, liver, kidney, heart, pancreas, intestine, abdominal wall, face/scalp, uterus, penis, and the like. The composition may further comprise an immunosuppressant, or may be administered in combination with an immunosuppressant. The composition, optionally in combination with an immunosuppressant may be administered during a transplantation operation, and/or induction phase) and/or after the transplantation operation for the long term (maintenance phase). When used as a combination, the composition and the immunosuppressant may be administered simultaneously or sequentially, and may be formulated into a single formulation or different formulations.

An aspect of the present disclosure also includes uses of a CHP, a pharmaceutically acceptable salt thereof, a stereoisomer, or a solvate thereof in the methods for suppressing an immune response; or in prolonging survival of a graft in an animal recipient and/or in the methods for prolonging survival of an animal recipient of a transplant. According to aspect, the CHP, a pharmaceutically acceptable salt thereof, a stereoisomer, or a solvate thereof, may be used in combination with an immunosuppressant. The composition, optionally in combination with an immunosuppressant may be administered during a transplantation procedure, and/or induction phase, and/or after the transplantation operation for the long term (maintenance phase). When used as a combination, the CHP, a pharmaceutically acceptable salt thereof, a stereoisomer, or a solvate thereof, and the immunosuppressant may be administered simultaneously or sequentially, and may be formulated into a single formulation or different formulations.

An aspect of the present disclosure also includes uses of a CHP, a pharmaceutically acceptable salt thereof, a stereoisomer, or a solvate thereof for use in manufacturing a medicament administered to prolong survival of a transplant in an animal recipient and/or in manufacturing a medicament administered to prolong survival of an animal recipient of a transplant. In the embodiment, the transplant includes organs, tissues, or cells of lung, liver, kidney, heart, pancreas, intestine, abdominal wall, face/scalp, uterus, penis, and the like. According to aspect, the CHP, a pharmaceutically acceptable salt thereof, a stereoisomer, or a solvate thereof, may be used in combination with an immunosuppressant. When used as a combination, the CHP, a pharmaceutically acceptable salt thereof, a stereoisomer, or a solvate thereof, and the immunosuppressant may be administered simultaneously or sequentially, and may be formulated into a single medicament or different medicaments. The medicament or medicaments may be administered during a transplantation procedure, and/or induction phase, and/or after the transplantation operation for the long term (maintenance phase).

Still another aspect of the present disclosure also includes a method of reducing an amount of an immunosuppressant administered to a subject during transplantation, and/or during induction phase, and/or during maintenance after transplantation, administering an effective amount of a CHP, a pharmaceutically acceptable salt thereof, a stereoisomer, or a solvate thereof to the subject. In the embodiment, a transplant to be transplanted to the subject includes organs, tissues, or cells of lung, liver, kidney, heart, pancreas, intestine, abdominal wall, face/scalp, uterus, penis, and the like. The CHP, a pharmaceutically acceptable salt thereof, a stereoisomer, or a solvate thereof, and the immunosuppressant may be administered simultaneously or sequentially, and may be formulated into a single formulation or different formulations. The formulation or formulations may be administered during a transplantation operation (induction phase) and/or after the transplantation operation for the long term (maintenance phase).

According to certain aspects, the present disclosure includes the following non-limiting exemplary embodiments.

Embodiment 1. A method for prolonging survival of a recipient of an allogenic transplant and/or prolonging survival of an allogenic transplant in a recipient thereof, comprising administering to the recipient an effective amount of cyclo-his-pro (CHP), a pharmaceutically acceptable salt thereof, stereoisomer, or a solvate thereof.

Embodiment 2. The method of Embodiment 1, further comprising providing the allogenic transplant, wherein the transplant is treated with cyclo-his-pro (CHP), a pharmaceutically acceptable salt thereof, stereoisomer, or a solvate thereof, before being transplanted into the recipient.

Embodiment 3. The method according to Embodiment 1, further comprising administering an immunosuppressant to the recipient.

Embodiment 4. The method according to Embodiment 1, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

Embodiment 5. The method according to Embodiment 1, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

Embodiment 6. The method according to Embodiment 3, wherein the administration of cyclo-his-pro (CHP), a pharmaceutically acceptable salt thereof, stereoisomer, or a solvate thereof and the administration of the immunosuppressant are carried out simultaneously, concurrently, or sequentially.

Embodiment 7. The method according to Embodiment 3, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

Embodiment 8. The method according to Embodiment 3, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

Embodiment 9. The method according to Embodiment 1, wherein the allogenic transplant is a tissue, an organ, or cells of lung, liver, kidney, pancreas, heart, intestine, abdominal wall, scalp, uterus, or penile.

Embodiment 10. The method according to Embodiment 3, wherein the allogenic transplant is a tissue, an organ, or cells of lung, liver, kidney, pancreas, heart, intestine, abdominal wall, scalp, uterus, or penile.

Embodiment 11. The method according to Embodiment 1, wherein the administration of cyclo-his-pro (CHP), a pharmaceutically acceptable salt thereof, stereoisomer, or a solvate thereof suppresses an immune reaction to the allogenic transplant in the recipient.

Embodiment 12. The method according to Embodiment 1, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.

Embodiment 13. A method for suppressing or decreasing an immune response to an allogenic transplant in a recipient thereof, for decreasing a rejection of an allogenic transplant in a recipient thereof, and/or for reducing an amount of an immunosuppressant administered during and/or after transplantation of an allogenic transplant to a recipient of the allogenic transplant, said method comprising administering an effective amount of cyclo-his-pro (CHP), a pharmaceutically acceptable salt thereof, stereoisomer, or a solvate thereof to the recipient.

Embodiment 14. The method of Embodiment 13, further comprising providing the allogenic transplant, wherein the transplant is treated with cyclo-his-pro (CHP), a pharmaceutically acceptable salt thereof, stereoisomer, or a solvate thereof, before being transplanted into the recipient.

Embodiment 15. The method according to Embodiment 13, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

Embodiment 16. The method according to Embodiment 13, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

Embodiment 17. The method according to Embodiment 13, wherein the administration of cyclo-his-pro (CHP), a pharmaceutically acceptable salt thereof, stereoisomer, or a solvate thereof and the administration of the immunosuppressant are carried out simultaneously, concurrently, or sequentially.

Embodiment 18. The method according to Embodiment 13, wherein the allogenic transplant is a tissue, an organ, or cells of lung, liver, kidney, pancreas, heart, intestine, abdominal wall, scalp, uterus, or penile.

Embodiment 19. The method according to Embodiment 13, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.

Embodiment 20. A composition for prolonging survival of a recipient of an allogenic transplant and/or prolonging survival of an allogenic transplant in a recipient thereof, comprising as an active ingredient an effective amount of cyclo-his-pro (CHP), a pharmaceutically acceptable salt thereof, stereoisomer, or a solvate thereof, and a pharmaceutically acceptable carrier.

Embodiment 21. The composition of Embodiment 20, wherein the allogenic transplant is treated with cyclo-his-pro (CHP), a pharmaceutically acceptable salt thereof, stereoisomer, or a solvate thereof, before being transplanted into the recipient.

Embodiment 22. The composition according to Embodiment 20, which is administered in combination with an immunosuppressant to the recipient.

Embodiment 23. The composition according to Embodiment 20, wherein the composition is administered to the recipient 0.5-18 hours or 18-36 hours prior to anesthesia for transplantation of the allogenic transplant in the recipient.

Embodiment 24. The composition according to Embodiment 20, wherein the composition is administered after transplantation to the recipient as a maintenance therapy.

Embodiment 25. The composition according to Embodiment 22, wherein the composition and the immunosuppressant to the recipient simultaneously, concurrently, or sequentially.

Embodiment 26. The composition according to Embodiment 22, wherein the composition is administered to the recipient 0.5-18 hours or 18-36 hours prior to anesthesia for transplantation of the allogenic transplant in the recipient.

Embodiment 27. The composition according to Embodiment 22, wherein the composition is administered after transplantation to the recipient as a maintenance therapy.

Embodiment 28. The composition according to Embodiment 20, wherein the allogenic transplant is a tissue, an organ, or cells of lung, liver, kidney, pancreas, heart, intestine, abdominal wall, scalp, uterus, or penile.

Embodiment 29. The composition according to Embodiment 20, wherein the allogenic transplant is a tissue, an organ, or cells of lung, liver, kidney, pancreas, heart, intestine, abdominal wall, scalp, uterus, or penile.

Embodiment 30. The composition according to Embodiment 20, wherein the administration of the composition suppresses an immune reaction to the allogenic transplant in the recipient.

Embodiment 31. The composition according to Embodiment 20, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.

Embodiment 32. A composition for suppressing or decreasing an immune response to an allogenic transplant in a recipient thereof, for decreasing a rejection of an allogenic transplant in a recipient thereof, and/or for reducing an amount of an immunosuppressant administered for and/or after transplantation of an allogenic transplant to a recipient of the allogenic transplant, said composition comprising as an active ingredient an effective amount of cyclo-his-pro (CHP), a pharmaceutically acceptable salt thereof, stereoisomer, or a solvate thereof, and a pharmaceutically acceptable carrier.

Embodiment 33. The composition of Embodiment 32, wherein the transplant is treated with cyclo-his-pro (CHP), a pharmaceutically acceptable salt thereof, stereoisomer, or a solvate thereof, before being transplanted into the recipient.

Embodiment 34. The composition according to Embodiment 32, wherein the composition is administered to the recipient 0.5-18 hours or 18-36 hours prior to anesthesia for transplantation of the allogenic transplant in the recipient.

Embodiment 35. The composition according to claim 32, wherein the composition is administered to the recipient after transplantation as a maintenance therapy.

Embodiment 36. The composition according to Embodiment 32, wherein the composition and the immunosuppressant are carried out simultaneously, concurrently, or sequentially.

Embodiment 37. The composition according to Embodiment 32, wherein the allogenic transplant is a tissue, an organ, or cells of lung, liver, kidney, pancreas, heart, intestine, abdominal wall, scalp, uterus, or penile.

Embodiment 38. The composition according to Embodiment 32, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.

Embodiment 39. A use of cyclo-his-pro (CHP), a pharmaceutically acceptable salt thereof, stereoisomer, or a solvate thereof in the manufacture of a medicine for (i) prolonging survival of a recipient of an allogenic transplant; (ii) prolonging survival of an allogenic transplant in a recipient thereof; (iii) suppressing or decreasing an immune response to an allogenic transplant in a recipient thereof; (iv) for decreasing a rejection of an allogenic transplant in a recipient thereof; and/or (v) for reducing an amount of an immunosuppressant administered during and/or after transplantation of an allogenic transplant to a recipient of the allogenic transplant.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a photograph of the kidney after reperfusion of the transplanted kidney. After reperfusion, the blood flowed smoothly and the kidney color was good.

FIG. 2 is a diagram showing an exemplary experimental flow of rat allogeneic kidney transplantation.

FIG. 3A and FIG. 3B show the survival rate of the kidney transplant in positive control group and the CHP administered group during the first 80-days (FIG. 3A) and the 1-year period (FIG. 3B) after the transplantation.

FIG. 4 is ultrasound pictures showing the smooth flow of blood in the transplanted body.

FIG. 5 shows histological images of transplanted kidneys in positive control group (Syngenic TPL) and in CHP treated group (TPL+CHP).

FIG. 6A-FIG. 6D show body weight (g), blood urea nitrogen (BUN) (mg/dL), creatine (mg/dL), Uproc/Crea ratio (mg/mg) changes during the 46 weeks period after the transplantation, respectively, in syngenic transplantation (Syngenic) and CHP-treated transplantation group (TPL+CHP).

FIG. 7A and FIG. 7B are graphs presenting a quantitative comparison of blood urea nitrogen (BUN) (FIG. 7A) and serum creatinine levels (FIG. 7B) in recipient rats five days post-transplantation. Individual data points represent the following groups: the syngeneic transplant recipients (Syngenic; n=3), kidney transplant recipients without CHP administration (TPL; n=5), and the CHP-treated transplant recipients (TPL+CHP; n=5).

FIG. 8 is the immunohistochemistry representative images of PAS and Nrf2 staining, with a scale bar indicating 1⁰⁰ m (at ×100 magnification). All animals were sacrificed at five days from post-kidney transplantation.

### DETAILED DISCLOSURE

### Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein. In an aspect, the word “about” as used in referring to a numerical value is intended to include 10%, 9%, 8%, 7%, 6%, 5%, 4%, 3%, 2%, or 1% variance from the numerical value(s).

The term “animal” used here includes all members of the animal kingdom including humans. The term “mammal” includes both human and non-human mammals. Similarly, the term “subject” includes both human and veterinary subjects.

The term “active agent” or “active ingredient” or “drug” or “medicament” or “formulation,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The phrase “consists essentially of” used herein with regard to a composition or formulation means that the composition or formulation contains the listed compound(s) as sole active ingredient(s) and may additionally contain a pharmaceutically acceptable inert additive(s), excipient(s), or carrier(s). Such inert additives, excipients, or carriers are known in the art.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging of survival of a recipient or a transplant as compared to expected survival if not receiving the treatment.

The phrase “pharmaceutically acceptable” additives, excipients, or carriers as used herein include those well known in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “pharmaceutically acceptable salt” refers to a salt that can be pharmaceutically used, among the substances having cations and anions coupled by electrostatic attraction. Typically, it may include metal salts, salts with organic bases, salts with inorganic acids, salts with organic acids, salts with basic or acidic amino acids or the like. Examples of the metal salts may include alkali metal salts (sodium salts, potassium salts, etc.), alkaline earth metal salts (calcium salts, magnesium salts, barium salts, etc.), aluminum salts or the like; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzylethylenediamine or the like; examples of the salts with inorganic acids may include salts with hydrochloric acid, hydrobromic acid, nitric acid, sulfuric acid, phosphoric acid, or the like; examples of the salts with organic acids may include salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid or the like; examples of the salts with basic amino acids may include salts with arginine, lysine, ornithine or the like; and examples of the salt with acidic amino acids include salts with aspartic acid, glutamic acid, or the like.

The term “therapeutically effective amount” or “effective amount,” or “effective dose,” as used herein, is the amount of the active agent(s) present in a composition described herein that is needed to provide a prolonging effect of a transplant or an animal recipient of a transplant. The precise amount will depend upon numerous factors, for example the specific activity of the composition, the delivery device employed, the physical characteristics of the composition, its intended use, as well as patient considerations such as severity of the disease state, patient cooperation, etc.

The terms “increased” or “increase” or “prolong” are used herein to generally mean an increase or prolong by a statically significant amount; in some embodiments, the terms “increased” or “increase” or “prolong” mean an increase or prolong of at least 10% as compared to a reference level (e.g., without the treatment or the administration described herein), for example an increase of at least about 10%, at least about 20%, or at least about 30%, or at least about 40%, or at least about 50%, or at least about 60%, or at least about 70%, or at least about 80%, or at least about 90% or up to and including a 100% increase (or prolong) or any increase (or prolong) between 10-100% as compared to a reference level. Other examples of “increase” or “prolong” include an increase or prolong of at least 2-fold, at least 5-fold, at least 10-fold, at least 20-fold, at least 50-fold, at least 100-fold, at least 1000-fold or more as compared to a reference level.

The term “suppress” or “suppressed” are used herein generally to mean that a progress of a disease or development of symptom(s) is slowed or decreased, compared to the absence of an intervention described herein.

The terms, “decreased” or “decrease” or “reduce” or “reduced” are used herein generally to mean a decrease by a statistically significant amount. In some embodiments, “decreased” or “decrease” means a reduction by at least 10% as compared to a reference level (e.g., without the treatment or the administration described herein), for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. In the context of a reduced amount of an immunosuppressant terms is meant a statistically significant decrease in such level. The reduction can be, for example, at least 10%, at least 20%, at least 30%, at least 40% or more, and is preferably down to a level accepted as within the range of normal for an individual without a given disease.

The term “induction phase” as used herein may be a period of preoperative, intraoperative (intubation, incision, transplantation, end of operation), and/or postoperative periods. The preoperative period may cover 0-3 days prior to, 1 day prior to, 2 day prior to, 3 days prior to, 0 day prior to, 24 hours prior to, 18 hours prior to, 15 hours prior to, 12 hours prior to, 6 hours prior to, 5 hours prior to, 4 hours prior to, 3 hours prior to, 2 hours prior to, 1 hour prior to, or 0.5 hour prior to the operation. Postoperative period may include about 0.5 day after, about 1 day after, about 2 days after, about 3 days after, about 4 days after, about 5 days after, about 6 days after, 7 about days after, about 10 days after, about 14 days after, about 1 month after, about 0-7 days after, about 0 day-1 month after, about 1 day 1-1 month after, 1 about −10 days after, about 0-14 days after, about 1-7 days after, about 1-10 days after, or about 1-14 days after the transplantation operation.

The term “induction therapy” as used herein may be an immunosuppressive therapy administered during the induction phase to reduce the risk of graft rejection. In general, induction strategies can include (i) a strategy employing high doses of conventional immunosuppressive agents, or (ii) a more commonly used strategy utilizing either T cell-depleting or interleukin (IL) 2 receptor-blocking antibodies in combination with lower doses of conventional agents.

The term “maintenance phase” as used herein may be a period after the induction phase.

The term “maintenance therapy” is an immunosuppressive therapy administered after the induction phase (when the risk of acute rejection decreases) to suppress immune response to the graft. The maintenance therapy is generally administered at a level lower than the induction therapy and decreases over the time to help lower the overall risk of infection and malignancy. Conventional maintenance regimens include a combination of immunosuppressive agents that differ by mechanism of action. This strategy minimizes morbidity and mortality associated with each class of agent while maximizing overall effectiveness.

The term “in combination with” includes the administration of two therapeutic agents (for example, CHP and an immunosuppressive agent (other than the CHP)) either simultaneously, concurrently or sequentially with no specific time limits. In one embodiment, both agents are present in the cell or in the patient's body at the same time or exert their biological or therapeutic effect at the same time. In one embodiment, the two therapeutic agents are in the same composition or unit dosage form. In another embodiment, the two therapeutic agents are in separate compositions or unit dosage forms.

The term “recipient” as used herein refers to an animal, including, but not limited to, a primate (e.g., human), cow, pig, sheep, goat, horse, dog, cat, rabbit, rat, or mouse. In embodiments, the term “recipient” refers to a mammalian subject, such as a human subject. In one embodiment, the recipient is a human.

### REJECTION OF TRANSPLANTS or GRAFT REJECTION

The disclosure in an aspect relates to the prevention and treatment of rejection, in particular of acute and/or chronic rejection, of a transplant by a recipient of the transplant.

The transplant includes tissues and organs of kidney, pancreas, liver, intestine, heart, lung, and/or vascularized composite allograft (VCA) transplant (e.g., uterus, abdominal wall, penis, face/scalp, other upper limb).

Hyperacute rejection occurs within minutes to hours after transplant and is due to preformed antibodies to the transplanted tissue antigens. It is characterized by hemorrhage and thrombotic occlusion of the graft vasculature. The binding of antibody to endothelium activates complement, and antibody and complement induce a number of changes in the graft endothelium that promote intravascular thrombosis and lead to vascular occlusion, the result being that the grafted organ suffers irreversible ischemic damage. Hyperacute rejection is often mediated by preexisting IgM alloantibodies, e.g., those directed against the ABO blood group antigens expressed on red blood cells. This type of rejection, mediated by natural antibodies, is the main reason for rejection of xenotransplants. Hyperacute rejection due to natural IgM antibodies is no longer a major problem with allografts because allografts are usually selected to match the donor and recipient ABO type.

Acute rejection is a process of vascular and parenchymal injury mediated by T cells, macrophages, and antibodies that usually begins after the first week of transplantation. T lymphocytes play a central role in acute rejection by responding to alloantigens, including MHC molecules, present on vascular endothelial and parenchymal cells. The activated T cells cause direct lysis of graft cells or produce cytokines that recruit and activate inflammatory cells, which cause necrosis. Both CD4+ and CD8+ cells may contribute to acute rejection. The destruction of allogeneic cells in a graft is highly specific and a hallmark of CD8+ cytotoxic T lymphocyte killing. CD4+ T cells may be important in mediating acute graft rejection by secreting cytokines and inducing delayed-type hypersensitivity-like reactions in grafts, with some evidence available that indicates that CD4+ T cells are sufficient to mediate acute rejection. Antibodies can also mediate acute rejection after a graft recipient mounts a humoral immune response to vessel wall antigens and the antibodies that are produced bind to the vessel wall and activate complement.

Chronic rejection (CR) or chronic allograft dysfunction (CAD) of solid organ allografts or transplants, regardless of type, develops slowly over a period of months or years. The process is characterized by luminal narrowing and occlusion of arteries and arterioles secondary to the proliferation of intimal smooth-muscle cells.

Several studies have shown that episodes of acute rejection, in particular severe, recurrent, and late episodes of rejection are major risk factors for chronic rejection.

For a transplant to be successful, the several modes of rejection must be overcome. Multiple approaches are utilized in preventing rejection. This includes administration of immunosuppressants, often several types to prevent the various modes of attack, e.g., inhibition of T-cell attack, antibodies, and cytokine and complement effects. Immunoadsorption of anti-HLA antibodies prior to grafting may reduce hyperacute rejection. Prior to transplantation the recipient or host may be administered anti-T cell reagents, e.g., the monoclonal antibody OKT3, Anti-Thymocyte Globulin (ATG), cyclosporin A, or tacrolimus (FK 506). Additionally, glucocorticoids and/or azathioprine (or other purine analogs) may be administered to the recipient or host prior to transplant. Drugs used to aid in preventing transplant rejection include, but are not limited to, ATG or anti-lymphocyte globulin (ALG), OKT3, daclizumab, basiliximab, corticosteroids, 15-deoxyspergualin, LF15-0195, cyclosporins, tacrolimus, purine analogs such as azathioprine, methotrexate, mycophenolate mofetil, 6-mercaptopurine, bredinin, brequinar, leflunamide, cyclophosphamide, sirolimus, everolimus, anti-CD4 monoclonal antibodies, CTLA4-Ig, rituxan, anti-CD154 monoclonal antibodies, anti-LFA1 monoclonal antibodies, anti-LFA-3 monoclonal antibodies, anti-CD2 monoclonal antibodies, and anti-CD45.

### Delayed Graft Function

Delayed graft function (DGF) is defined as the need for dialysis during the first week after transplantation, and is the most frequent early posttransplant complication. DGF is one of the main predictors of poor graft survival in cadaver donor renal transplantation. DGF is common, affecting between 20-60 in 100 of kidneys transplanted from deceased donors. DGF is a manifestation of acute kidney injury (AKI) with attributes unique to the transplant process. And, DGF is a major obstacle for allograft survival as it can be compounded by acute rejection and chronic allograft nephropathy (CAN).

### CHP and Compositions Containing CHP

Cyclo histidine-proline (Cyclo His-Pro, CHP) is a naturally-generating cyclic dipeptide that is structurally related to thyrotropin-releasing hormone (TRH). The cyclo histidine-proline (CHP) is a peptide inherent in animal and human tissues and body fluids. The CHP is found in blood, semen, gastrointestinal tract, urine, etc., and in particular is a metabolite rich in prostate. The cyclo histidine-proline (CHP) has been known to have a variety of physiological functions such as anti-diabetes, anti-obesity, anti-inflammatory and antioxidant effects.

According to an aspect of the disclosure, CHP or a pharmaceutically acceptable salt thereof, a stereoisomer, or a solvate thereof is used as an active agent or active ingredient for the methods and compositions. It should be understood that the term “CHP” used in the present disclosure sometimes to collectively refer to the CHP, a pharmaceutically acceptable salt thereof, a stereoisomer, or a solvate thereof.

According to the embodiments described herein, the CHP is used to broadly include CHP of the formulas above, a pharmaceutically acceptable salt thereof, a stereoisomer, a solvate thereof, unless specified otherwise.

A cyclo (−His-Pro) (CHP) is illustrated below:

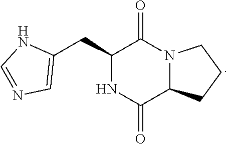 [SMILES: O=C1N[C@@H](Cc2cnc[nH]2)C(=O)N2CCC[C@@H]12]

As a non-limiting example of a CHP solvate, a CHP monohydrate is illustrated below:

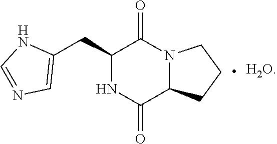

In one embodiment, the CHP is substantially pure.

In an embodiment, the CHP is a CHP hydrate. In still another embodiment, the CHP hydrate is characterized by an XRPD diffractogram comprising peaks at about 17±0.2° and about 27.3±0.2° in 20. One embodiment of substantially pure CHP hydrate is characterized by an X-ray powder diffractogram comprising at least three peaks chosen from the following list: 13.7, 17, 18.1, 20.2 and 27.3 degrees (+0.2° in 20). Another embodiment is characterized by an XRPD diffractogram comprising at least two peaks chosen from the following list: 10, 13.7, 17, 18.1, 20.2 and 27.3 degrees (+0.2° in 20). CHP hydrate, as one of a CHP solvate, can be made by a process described in U.S. application Ser. No. 16/448,083, of which content is incorporated herein by reference, in its entirety.

CHP synthesized from different biochemical sources, including histidine-proline-rich glycoprotein. High levels of CHP are present in many food sources, and are readily absorbed in the gut without chemical or enzymatic destruction.

A composition suitable for reducing or suppressing rejection of a transplant in a recipient animal, treating the recipient animal to prolong survival of the recipient animal, and/or treating a transplant (before being transplanted) to prolong survival of the transplant in the recipient animal may comprise or consist essentially of a CHP, a pharmaceutically acceptable salt thereof, a stereoisomer, or a solvate thereof, and may comprise a pharmaceutically acceptable carrier or excipient. The composition may be administered or employed alone for reducing or suppressing rejection of a graft in a recipient animal, treating the recipient animal to prolong survival of the recipient animal, treating the recipient animal to prolong survival of the graft in the recipient animal, and/or treating a transplant to prolong survival of the transplant in the recipient animal. The composition may be administered or employed in combination with an immunosuppressant (that is different from the CHP) for reducing or suppressing rejection of a graft in a recipient animal, treating the recipient animal to prolong survival of the recipient animal, and/or treating a graft to prolong survival of the transplant in the recipient animal, and/or delaying or suppressing graft loss, and/or preventing or treating delayed graft function, and/or reducing the amount of the immunosuppressant administered in the induction phase and/or the maintenance phase. The composition comprising or consisting essentially of the CHP and the immunosuppressant (that is different from the CHP) may be administered simultaneously, concurrently, or sequentially.

The composition may be employed to reduce or decrease the amount of an immunosuppressant (other than the CHP) administered to a transplant recipient during or for the transplantation or for maintenance (after the transplant). The composition comprising or consisting essentially of the CHP and the immunosuppressant (other than the CHP) may be administered simultaneously, concurrently, or sequentially.

In one embodiment, a CHP may be present in a composition in amount ranging from about 0.5 to about 10000 mg, from about 1 to 5000 mg, from about 1 to 2000 mg, or from about 10 to about 1000 mg. In another embodiment, the amount of CHP present in the administered pharmaceutical composition may range from about 5 to about 3000 mg, from about 50 to about 2000 mg, from about 100 to about 2000 mg, from about 50 to about 1000 mg, from about 100 to about 1000 mg, from about 150 to about 2000 mg, from about 200 to about 1000 mg, from about 50 to about 800 mg, from about 100 to about 700 mg, from about 50 to about 600 mg, or from about 100 to about 1500 mg, as calculated in term of anhydrous CHP. The composition may be a pharmaceutical composition, foodstuff, or a dietary supplement. In particular embodiment, the composition is a pharmaceutical composition.

In another embodiment, the composition is suitable for treatment of a transplant to prolong the survival of the transplant in a recipient animal. The composition may be a liquid in which the transplant is infused. In an embodiment, the concentration of liquid formulation is from about 1 mg/liter to about 200 mg/ml, from about 5 mg/ml to about 150 mg/ml, from about 10 mg/ml to about 100 mg/ml. In another embodiment, the concentration of the liquid formulation is about 1 mg/liter, about 2 mg/liter, about 3 mg/liter, about 4 mg/liter, about 5 mg/liter, about 6 mg/liter, about 7 mg/liter, about 8 mg/liter, about 9 mg/liter, about 10 mg/liter, about 11 mg/liter, about 12 mg/liter, about 13 mg/liter, about 14 mg/liter, about 15 mg/liter, about 20 mg/liter, about 25 mg/liter, about 30 mg/liter, about 35 mg/liter, about 40 mg/liter, about 45 mg/liter, about 50 mg/liter, about 55 mg/liter, about 60 mg/liter, about 65 mg/liter, about 70 mg/liter, about 75 mg/liter, about 80 mg/liter, about 85 mg/liter, about 90 mg/liter, about 95 mg/liter, about 100 mg/liter, about 110 mg/liter, about 120 mg/liter, about 130 mg/liter, about 140 mg/liter, about 150 mg/liter, about 160 mg/liter, about 170 mg/liter about 180 mg/liter, about 190 mg/liter, or about 200 mg/liter.

In still another embodiment, the composition is suitable for administering to a recipient animal who will receive, receives, or has received a transplant. The composition may comprise a known immunosuppressant. Alternatively, CHP and an immunosuppressant may be administered in separate formulations, simultaneously or sequentially. In another embodiment, the composition may consist essentially of CHP. In still another embodiments, the composition, which comprise CHP alone or optionally with an immunosuppressant, may further comprise another therapeutically active agent. Alternatively, the composition which comprises CHP alone or optionally with an immunosuppressant may be administered or used separately from another composition comprising the another therapeutically active agent.

In some embodiments, the another therapeutically active agent may include a biomolecule, bioactive agent, small molecule, drug, prodrug, drug derivative, protein, peptide, vaccine, adjuvant, imaging agent (e.g., a fluorescent moiety), polynucleotide or a metal. In yet another embodiment, the active agent is a metal element, metal cation, a metal complex, or a metal compound wherein the metal can be copper, zinc, magnesium, manganese, iron, cobalt, chromium, or a combination thereof. In an embodiment, the metal is zinc and a zinc compound may be zinc gluconate, zinc acetate, zinc sulfate, zinc picolinate, zinc orotate, or zinc citrate. In another embodiment, the metal is magnesium and a magnesium compound such as magnesium oxide, magnesium citrate, magnesium chloride, magnesium glycinate, magnesium biglycinate, magnesium aspartate, magnesium lactate, or magnesium chloride can be employed. In another embodiment, the metal is manganese and a manganese compound may include manganese amino acid chelates (e.g., manganese bisglycinate chelate, manganese glycinate chelate, manganese aspartate, manganese gluconate, manganese picolinate, manganese sulfate, manganese citrate, or manganese chloride. In an embodiment, the metal is copper and a copper compound may include a cupric oxide, cupric sulfate, copper amino acid chelates, and copper gluconate. In still another embodiment, the metal is iron and iron may exist in various forms such as ferrous and ferric iron salts (for example, ferrous sulfate, ferrous gluconate, ferric citrate, or ferric sulfate cobalt). In an embodiment, the metal is cobalt and a cobalt compound may include cobalt acetate, cobalt sulfate, cobalt picolinate, cobalt orotate, or cobalt citrate. In an embodiment, the metal is chromium and a chromium compound may include chromium chloride, chromium nicotinate, chromium picolinate, high-chromium yeast, or chromium citrate.

In exemplary embodiments, the pharmaceutical composition of the embodiments can be administered in a variety of ways, including orally, topically, parenterally, intravenously, intradermally, colonically, rectally, intramuscularly or intraperitoneally.

The pharmaceutical composition may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The formulation may take such forms as suspensions, solutions or emulsions in oily or aqueous vehicles, and may contain agents such as suspending, stabilizing and/or dispersing agents.

For example, a parenteral preparation may be a sterile injectable solution or suspension in a nontoxic parenterally acceptable diluent or solvent. Among the acceptable vehicles and solvents that may be employed are water, 0.9% saline solution, or other suitable aqueous media.

In one embodiment, the concentration of the intravenous solution formulation is from about 1 mg/liter to about 200 mg/ml, from about 5 mg/ml to about 150 mg/ml, from about 10 mg/ml to about 100 mg/ml. In another embodiment, the concentration of the intravenous solution formulation is about 1 mg/liter, about 2 mg/liter, about 3 mg/liter, about 4 mg/liter, about 5 mg/liter, about 6 mg/liter, about 7 mg/liter, about 8 mg/liter, about 9 mg/liter, about 10 mg/liter, about 11 mg/liter, about 12 mg/liter, about 13 mg/liter, about 14 mg/liter, about 15 mg/liter, about 20 mg/liter, about 25 mg/liter, about 30 mg/liter, about 35 mg/liter, about 40 mg/liter, about 45 mg/liter, about 50 mg/liter, about 55 mg/liter, about 60 mg/liter, about 65 mg/liter, about 70 mg/liter, about 75 mg/liter, about 80 mg/liter, about 85 mg/liter, about 90 mg/liter, about 95 mg/liter, about 100 mg/liter, about 110 mg/liter, about 120 mg/liter, about 130 mg/liter, about 140 mg/liter, about 150 mg/liter, about 160 mg/liter, about 170 mg/liter about 180 mg/liter, about 190 mg/liter, or about 200 mg/liter.

In another embodiment, the pharmaceutical composition may be formulated into a diffusion (slow drip) formulation or an intravenous bolus injection.

In yet another embodiment, the pharmaceutical composition may be administered orally or formulated for oral administration. Administration may be via immediate release tablets and capsule or enteric-coated tablets or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, sterile injectable solutions and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, hydroxypropyl cellulose, polyvinylpyrrolidone, cellulose, USP or sterile water, syrup base and methyl cellulose. The formulations can additionally include: lubricating agents such as talc, magnesium stearate, and stearic acid; wetting agents; emulsifying and suspending agents; preserving agents such as methyl and propylhydroxy-benzoates; sweetening agents; and flavoring agents.

In some embodiments, the pharmaceutical compositions are formulated in a unit dosage form. The term “unit dosage forms” refers to physically discrete units suitable as unitary dosages for human subjects and other mammals, each unit containing a predetermined quantity of active material (therapeutically effective amount) calculated to produce the desired therapeutic effect, in association with a suitable pharmaceutical excipient (e.g., a tablet, capsule, ampoule). The compounds are generally administered in a pharmaceutically effective amount. In some embodiments, each dosage unit contains from about 1 mg to about 100 mg of a CHP compound. In some embodiments, each dosage unit contains from about 2 mg to about 60 mg, from about 3 mg to about 50 mg, from about 4 mg to about 40 mg, from about 5 mg to about 30 mg, from about 6 mg to about 20 mg, from about 8 mg to about 15 mg, or from about 8 mg to about 10 mg of a CHP compound.

In other embodiments, each dosage unit contains about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 30 mg, about 40 mg, about 50 mg, about 60 mg, about 70 mg, about 80 mg, about 90 mg, or about 100 mg of a CHP compound.

For preparing solid compositions such as tablets, the active principle ingredient is mixed with a pharmaceutical excipient to form a solid mixed-blend composition containing a homogeneous mixture of a compound of the present disclosure. When referring to these mixed-blend compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be powder-coated or otherwise compounded to provide a dosage form affording the advantage of prolonged action or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. In one embodiment, the film coating is a polyvinyl alcohol-based coating.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

Suitable excipients include binders, fillers, disintegrants, lubricants, antioxidants, chelating agents, and color agents.

In yet another embodiments, the compositions disclosed herein may be a food or a dietary supplement. The food composition or dietary supplement composition may contain a pharmaceutically acceptable excipient as described herein with respect to pharmaceutical compositions.

### Methods

According to an aspect of the present disclosure, a method of suppressing an immune response comprising or consisting essentially of administering an effective amount of a cyclo histidine-proline (cyclo His-Pro or CHP), a pharmaceutically acceptable salt thereof, a stereoisomer, or a solvate thereof to an animal in need of such treatment is disclosed. In the embodiment, the immune response may include acute rejection and/or chronic rejection of transplant by an animal recipient.

In another embodiment, the present disclosure provides a method of suppressing an immune response to a transplanted organ, tissue or cell comprising or consisting essentially of administering an effective amount of a CHP, a pharmaceutically acceptable salt thereof, a stereoisomer, or a solvate thereof to a mammal in need thereof. In the embodiment, the transplanted organ, tissue or cell includes organs, tissues, or cells of lung, liver, kidney, heart, pancreas, intestine, abdominal wall, face/scalp, uterus, penis, and the like. In the embodiment, the immune response may include acute rejection and/or chronic rejection of transplant by an animal recipient.

Still another aspect of the present disclosure provides a method of prolong a survival of transplant in a recipient animal comprising or consisting essentially of treating the transplant with a composition comprising a CHP, a pharmaceutically acceptable salt thereof, a stereoisomer, or a solvate thereof. In the embodiment, the transplant includes organs, tissues, or cells of lung, liver, kidney, heart, pancreas, intestine, abdominal wall, face/scalp, uterus, penis, and the like.

Yet still another aspect of the present disclosure provides a method of prolong a survival of an animal recipient of transplant comprising or consisting essentially of administering to the animal recipient an effective amount of a composition comprising a CHP, a pharmaceutically acceptable salt thereof, a stereoisomer, or a solvate thereof. In the embodiment, the transplant includes organs, tissues, or cells of lung, liver, kidney, heart, pancreas, intestine, abdominal wall, face/scalp, uterus, penis, and the like.

With regard to any of the above-discussed methods, CHP may be administered to the recipient in an amount from about 0.001 to about 3000 mg/kg. In some embodiments, the effective amount of the CHP may be about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg. The amounts are based on the amount of anhydrous CHP.

With regard to any of the above-discussed methods, CHP may be administered to the animal recipient in an amount from about 1 to about 3000 mg/day. In some embodiments, the effective amount of the CHP may be in a range of about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day, or 2900-3000 mg/day. The amounts are based on the amount of anhydrous CHP.

With regard to any of the above-discussed methods, the methods may further comprise administering an immunosuppressant described below. The immunosuppressant may be contained in a same or different formulation from a composition containing a CHP. When an immunosuppressant is contained in a separate formulation, the immunosuppressant may be administered simultaneously or sequentially with CHP.

With regard to the above-discussed administration methods, the recipient may be administered with the pharmaceutical composition described herein prior to, during, and/or after transplant procedure, at the above-described dose by one or more of administration routes discussed above. In some embodiments, the CHP may be administered to a recipient 0.5-24 hours, 0.5-18 hours, 0.5-12 hours, 0.5-9 hours, 0.5-6 hours, or 0.5-3 hours, prior to the transplantation by a first route of administration. In some embodiment, the CHP may be administered starting from 0.5-3 hours prior to anesthesia and ending at 0-12 hours, 1-8 hours, or 2-6 hours after awake from anesthesia. The recipient may be further administered by a second route of administration with the CHP for 1-30 days. The first route of administration and the second route of administration may be the same or different from each other. In non-limiting exemplary embodiment, the first route of administration may be intravenous and the second round of administration may be intraperitoneal, intravenous, oral, of a combination thereof.

With regard to the above-discussed administration methods, the methods may further comprise administering CHP to the transplant's donor before extracting the transplant from the donor, and/or providing the transplant extracted from the donor with CHP before being transplanted to the recipient.

### Immunosuppressant

Various drugs utilized to delay graft rejection (i.e., to prolong their survival) work in a variety of ways. Immunosuppressive agents are widely used.

Examples of biological immunosuppressants employed in induction therapy may include, but are not limited to, various polyclonal antibody or monoclonal antibodies.

ALG (anti-lymphocyte globulin), ATG (anti-T-lymphocytes globulin), Thymoglobulin (TG)-anti-thymocytes globulin (obtained by immunization of rabbits), Lymphoglobulin (LG)-anti-thymocytes globulin (obtained by immunization of horses), and the like. These polyclonal antibodies show the cytotoxicity of antibodies directed against a variety of T-cell markers and depletion of the lymphocyties from the peripheral blood. Polyclonal antibodies can produce leukopenia and thrombocytopenia.

Muromonab (OKT3) is a lymphocyte-depleting monoclonal antibody, which is produced by the hybridization of murine antibody-secreting B lymphocytes with a nonsecreting myeloma cell line. In the first and second day of treatment with OKT3, potentially life-threatening adverse reactions may occur and this is the reason why it is now used only when thymoglobulin is contraindicated because of leukopenia or thrombocytopenia. lemtuzumab (Campath 1H) is a recombinant DNA-derived humanized monoclonal antibody directed against the cell surface glycoprotein CD52. Humanized anti-CD25 monoclonal antibodies include Basiliximab (SIMULECT™) and Daclizumab (ZENAPAX™). These antibodies are targeted against the alpha chain of the IL-2 receptor and the IL-2 mediated responses are blocked. Rituximab (RITUXAN™, MABTHERA™) is a monoclonal anti-CD20 antibody, targeted against the CD20-antigen on B lymphocytes. Efalizumab (RAPTIVA™), which is a humanized CD11a-specific IgG1, targeted against lymphocyte-associated function-1 (LFA-1) molecule; Alefacept (AMEVIVE™) which is a humanized LFA-3-IgG1 fusion protein that binds to CD2 in the T lymphocyte and interferes with T-Cell activation; Bortezomib (VELCADE™) which is a proteosomal inhibitor and suppresses the T-cell function, and like, are also used in the induction phase.

During the maintenance phase, various immunosuppressant may be used, which may include, but are not limited to, calcineurin inhibitors (CNI) (e.g., cyclosporine (CsA) and tacrolimus (Tac)), mycophenolate mofetil (MMF), mycophenolic Acid (MPA), mTOR inhibitors (e.g., sirolimus (RAPAMUNE™), everolimus (CERTICAN™)), corticosteroids (e.g., prednison and methylprednisolone), and the like. These immunosuppressants may be administered alone or in combinations.

Inhibitors of purine or pyrimidine biosynthesis are also used to inhibit transplant rejection (or graft rejection). These prevent DNA synthesis and thereby inhibit cell division including the ability of T cells to divide. The result is the inhibition of T cell activity by preventing the formation of new T cells. Inhibitors of purine synthesis include azathioprine, methotrexate, mycophenolate mofetil (MMF) and mizoribine (MZB, BREDININ™). Inhibitors of pyrimidine synthesis include brequinar sodium and leflunomide. Cyclophosphamide is an inhibitor of both purine and pyrimidine synthesis.

Many other drugs and methods for delaying allotransplant rejection are known to and used by those of skill in the art. Administration to the recipient of an inhibitor (blocker) of the CD40 ligand-CD40 interaction and/or a blocker of the CD28-B7 interaction has been proposed (U.S. Pat. No. 6,280,957). Published PCT patent application WO 01/37860 teaches the administration of an anti-CD3 monoclonal antibody and IL-5 to inhibit the Th1 immune response. Published PCT patent application WO 00/27421 teaches a method for prophylaxis or treatment of corneal transplant rejection by administering a tumor necrosis factor-α antagonist. U.S. Patent Application Publication US 2003/0180301 discloses a treatment of chronic transplant rejection by administering an antagonist of TGF-β.

Side effects of CNI include, for example, nephrotoxicity, enhancement of early post-transplant graft dysfunction, dose related reversible renal vasoconstriction, chronic interstitial fibrosis, acute microvascular disease, hypertension, gastrointestinal dysfunction, norexia, nausea, vomiting, diarrhea and abdominal discomfort, hair loss, and the like. Adverse effects of MMR (CELLCEPT™) and enteric-coated MPA (MYFORTIC™) include, for example, gastro-intestinal adverse effects such as diarrhea, varying degrees of nausea, bloating, dyspepsia, vomiting, frank esophagitis, gastritis. Most of these symptoms respond to the reduction of drug dosage. Adverse effects of sirolimus may include, for example, tubulotoxic, hypokaliemia, hypomagnesmia, proteinuria, nephritis syndrome, or the like. Corticosteroids inhibit the dendritic cells, inhibit the transcription of cytokines genes and all the stages of the T-cell activation; and the nonspecific immunosuppressive effects are lymphopenia.

The following non-limiting examples are illustrative of the present disclosure.

### EXAMPLES

### Example 1: Allogeneic Kidney Transplantation in Rats

Donor surgery: After removing abdominal hair from donor rat under general anesthesia, the surgical area was disinfected and covered with surgical cloth. After a midline incision in the abdominal wall, one kidney was exposed, and 8 iu/kg of heparin was administered intravenously, followed by nephrectomy.

Table Surgery: Removed unnecessary tissue to anastomosis the artery, vein, and ureter of the extracted kidney to the blood vessel and ureter of the recipient, and covered and stored with gauze soaked in saline at 4° C.

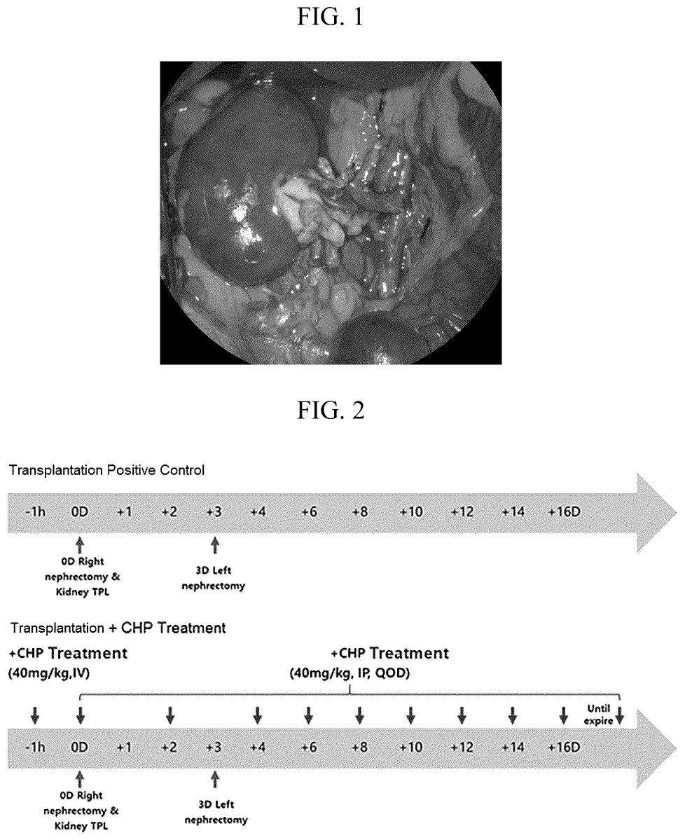

Recipient surgery: After removal of abdominal hair of recipient rat under general anesthesia, the surgical area was disinfected and covered with surgical cloth. After a midline incision in the abdominal wall, the right kidney was exposed, the kidney was resected, and the donor renal artery and vein were anastomized end to side to the abdominal vena cava. The ureter of donor's kidney was sutured after incising the bladder. After confirming that the perfusion of the kidney was good (FIG. 1), the abdominal wall was closed, and the animal was placed on a blanket at 37° C. to recover.

### Example 2. Survival Rate of Control Group and CHP Administration Group

As a minor MHC incompatible model, a kidney transplantation model F344 rats were used as donors and LEWIS rats were used as recipients. After resection of the recipients' right kidney on Day 0 (DO), the donors' kidney was transplanted into the recipients. The recipients' left kidney was resected on Day 3 (D3). The transplantation and CHP treatment schedule is shown in FIG. 2. The CHP was intravenous administered at a dose of CHP 40 mg/kg 1 hour before kidney transplantation, followed by intraperitoneal administrations of CHP 40 mg/kg three times a week.

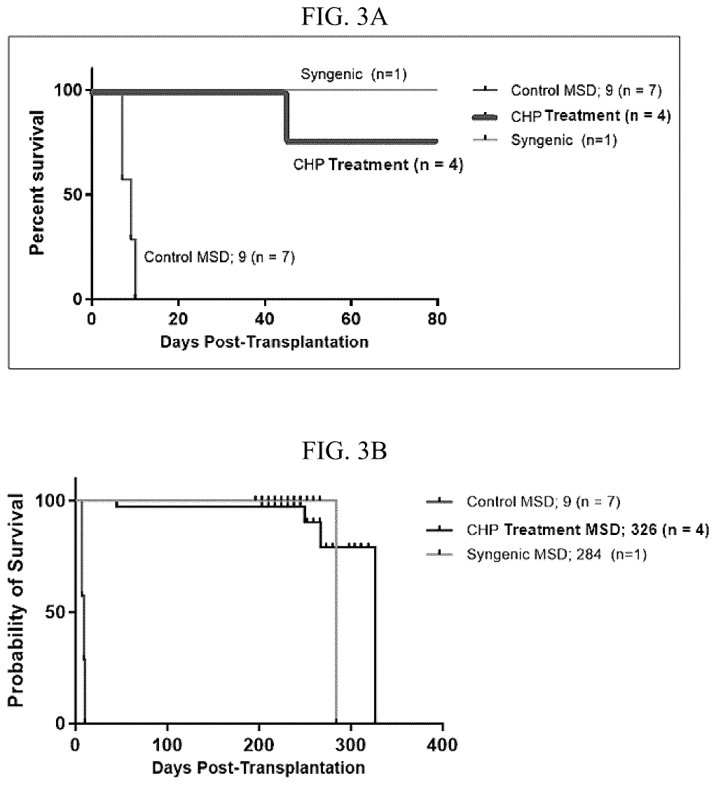

The mean survival day (SMD) of 7 control animals not administered with CHP was 9 days, and the SMD of 4 animals administered with CHP was 326 days (n=4) (FIGS. 3A and 3B). It has been confirmed that the recipient animals treated with CHP 1 day before the transplantation procedure, on the operation day, and the post-operative period (i.e., induction phase) and the maintenance period showed remarkably prolonged survival rate.

The results show that CHP could significantly prolong the average survival of recipients.

### Example 3. Representative Ultrasound Pictures of Control Group, CHP Administration Group and Inbred (Syngenic) Transplant Group

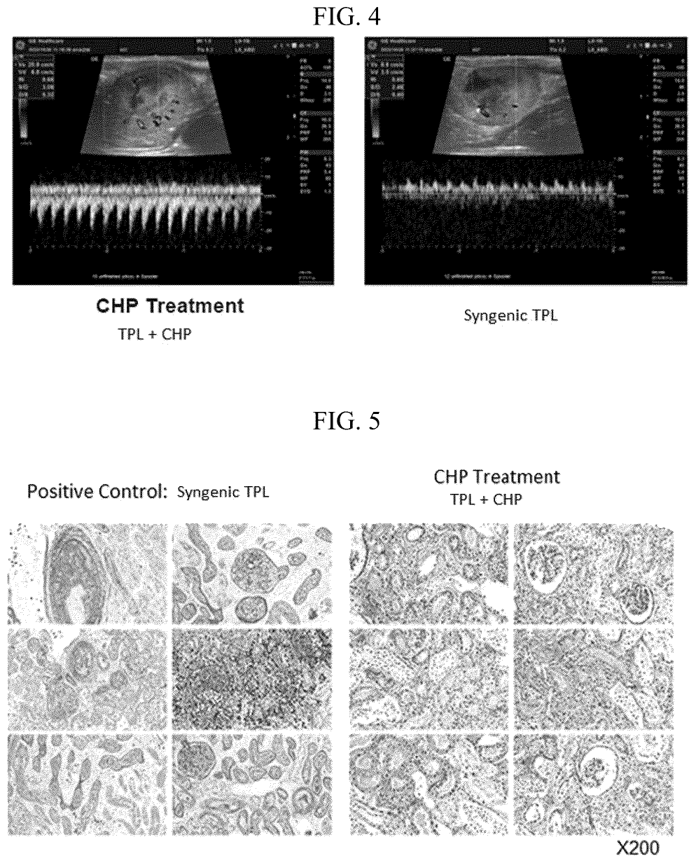

In the control group, CHP administration group (F344 to LEWIS rats), and inbred (LEWIS to LEWIS rats) kidney transplantation model, ultrasound was performed once a week to check the smooth flow of blood to the transplanted kidney. As can be seen from the images of FIG. 4, blood flow to the kidney was smooth in the CHP treatment group indicating the transplanted kidney functions normally, whereas the kidney transplant recipient without CHP treatment (“Syngenic TPL”) shows significantly reduced blood flow to the kidney. When the function of the transplanted kidney was smooth, the blood flow to the kidney was confirmed by color Doppler, and the RI index also showed a value between 0.5 and 0.7. When the function of the transplanted kidney decreased, the blood flow to the kidney decreased, and the RI index also showed a value of 0.7 or higher.

### Example 4. Representative Graft Tissue Findings of Control Group and CHP Administration Group

In the CHP-treated group (TPL+CHP), not only did focal glomerular necrosis and severe tubulointerstitial changes decrease, but also findings accompanied by borderline changes suggestive of acute cell-mediated rejection and microvascular inflammation rapidly decreased.

In terms of overall renal histology, important changes such as acute tubular injury, i.e., acute inflammatory cell infiltration around the tubule or glomerulus, formation of thrombosis in capillaries, and inflammation of the arterial wall were observed in the CHP-treated group. These phenomena are believed to be an effect of CHP that can suppress the acute rejection reaction that occurs within days after transplantation. In FIG. 5, graft kidney tissue findings on the 7th day of rejection in the left positive control group (Syngenic TPL) and on the 45th day in the CHP treated group (TPL+CHP).

### Example 5. Evaluation of Graft Function

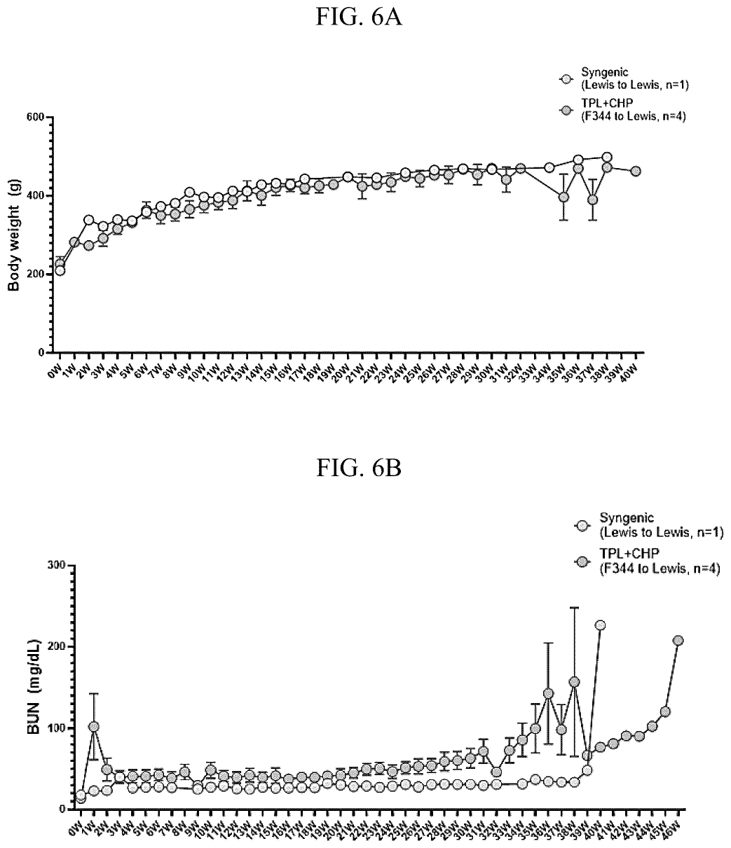

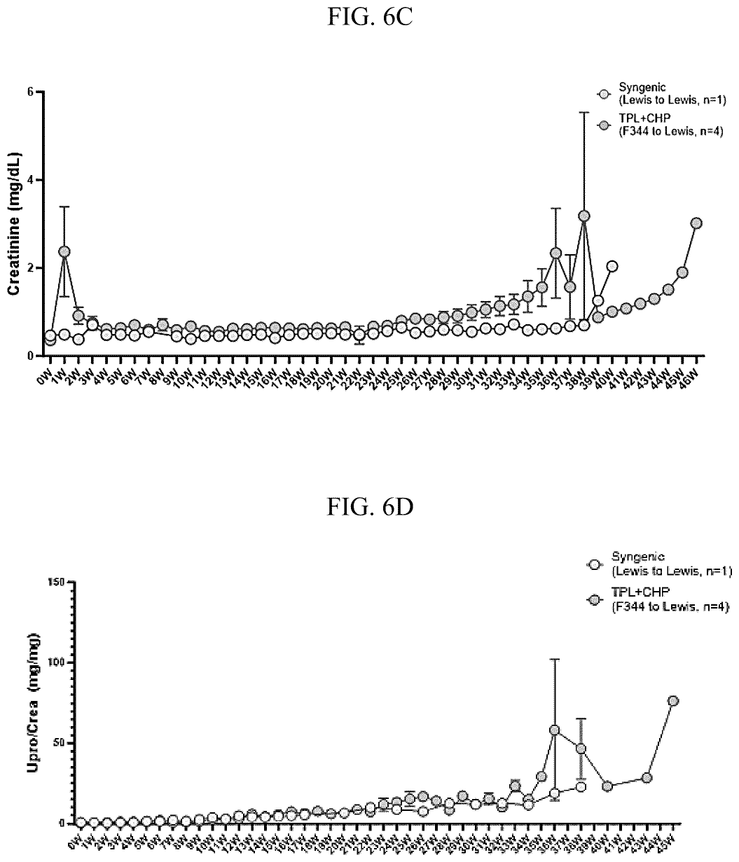

Body weight (g), blood urea nitrogen (BUN) (mg/dL), creatine (mg/dL), Uproc/Crea ratio (mg/mg) of the CHP-treated animal group (TPL+CHP) and syngenic group (Syngenic) were measured weekly during the 46 weeks period after the transplantation, and the results are shown in FIG. 6A-FIG. 6D, respectively. Serum creatinine of the inbred negative control group (Syngenic) was 0.71 mg/dL at 3 weeks, and a similar value was observed in the CHP treated group (TPL+CHP) with a value of 0.82 mg/dL.

The results show that the grafted organ of the CHP-treated animals were fully function after the transplantation during the entire period of the survival of the recipient animals.

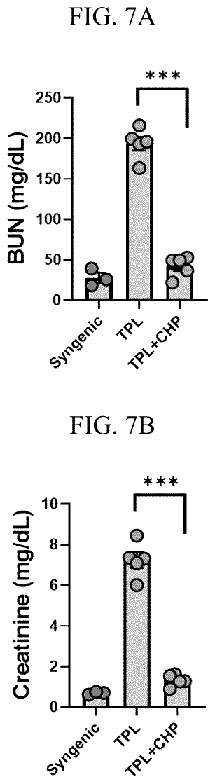

FIG. 7 shows graphs presenting a quantitative comparison of BUN and serum creatinine levels in recipient rats five days post-transplantation. Individual data points represent the following groups: the syngeneic transplant recipients (Syngenic; n=3), transplant recipients without CHP administration (TPL; n=5), and the CHP-treated transplant recipients (TPL+CHP; n=5). Recipients treated with CHP exhibited a significant reduction in both BUN (from 193.5±8.54 to 41.98±5.69; p-value <0.0001) and serum creatinine levels (from 7.23±0.39 to 1.32±0.13; p-value <0.0001). This significant improvement in key renal function indicators highlights a promising advancement in post-transplant care, reducing the reliance on immunosuppressive therapies, and prolonging recipient survival and enhancing graft survival.

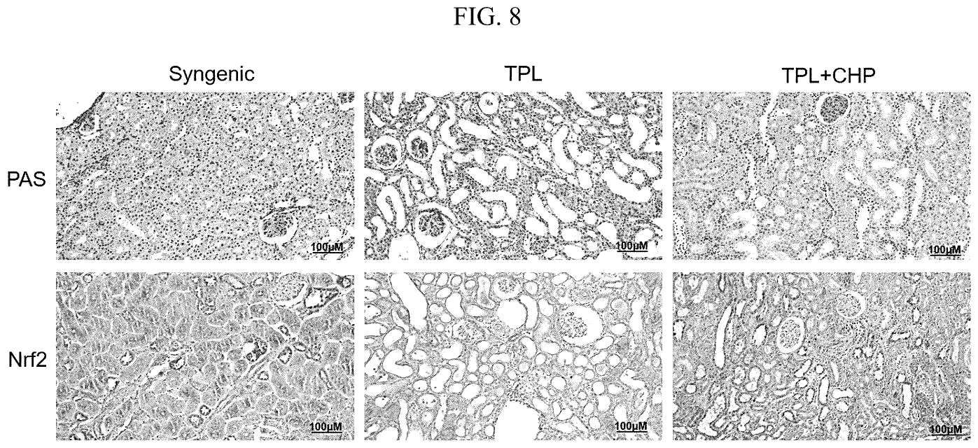

FIG. 8 shows the immunohistochemistry representative images of PAS and Nrf2 staining, with a scale bar indicating 100 m (at ×100 magnification). All animals were sacrificed at five days from post-kidney transplantation. The administration of CHP to the transplant recipient group decreased tubular atrophy, tubulitis, endothelialitis, leukocyte infiltration, while increasing the level of antioxidative stress marker Nrf2 (Nuclear factor erythroid-2-related factor). These findings highlight the potential of CHP to exert protective (e.g., anti-inflammatory and antioxidative) effects in the transplantation rat model.

While the invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method for prolonging survival of a recipient of an allogenic transplant and/or prolonging survival of an allogenic transplant in a recipient thereof, comprising administering to the recipient an effective amount of cyclo-his-pro (CHP), a pharmaceutically acceptable salt thereof, stereoisomer, or a solvate thereof.

2. The method of claim 1, further comprising providing the allogenic transplant, wherein the transplant is treated with cyclo-his-pro (CHP), a pharmaceutically acceptable salt thereof, stereoisomer, or a solvate thereof, before being transplanted into the recipient.

3. The method according to claim 1, further comprising administering an immunosuppressant to the recipient.

4. The method according to claim 1, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

5. The method according to claim 1, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

6. The method according to claim 3, wherein the administration of cyclo-his-pro (CHP), a pharmaceutically acceptable salt thereof, stereoisomer, or a solvate thereof and the administration of the immunosuppressant are carried out simultaneously, concurrently, or sequentially.

7. The method according to claim 3, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

8. The method according to claim 3, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

9. The method according to claim 1, wherein the allogenic transplant is a tissue, an organ, or cells of lung, liver, kidney, pancreas, heart, intestine, abdominal wall, scalp, uterus, or penile.

10. The method according to claim 3, wherein the allogenic transplant is a tissue, an organ, or cells of lung, liver, kidney, pancreas, heart, intestine, abdominal wall, scalp, uterus, or penile.

11. The method according to claim 1, wherein the administration of cyclo-his-pro (CHP), a pharmaceutically acceptable salt thereof, stereoisomer, or a solvate thereof suppresses an immune reaction to the allogenic transplant in the recipient.

12. The method according to claim 1, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.

13. A method for suppressing or decreasing an immune response to an allogenic transplant in a recipient thereof, for decreasing a rejection of an allogenic transplant in a recipient thereof, and/or for reducing an amount of an immunosuppressant administered during and/or after transplantation of an allogenic transplant to a recipient of the allogenic transplant, said method comprising administering an effective amount of cyclo-his-pro (CHP), a pharmaceutically acceptable salt thereof, stereoisomer, or a solvate thereof to the recipient.

14. The method of claim 13, further comprising providing the allogenic transplant, wherein the transplant is treated with cyclo-his-pro (CHP), a pharmaceutically acceptable salt thereof, stereoisomer, or a solvate thereof, before being transplanted into the recipient.

15. The method according to claim 13, wherein the administration of cyclo-his-pro (CHP), a pharmaceutically acceptable salt thereof, stereoisomer, or a solvate thereof begins 0.5-18 hours or 18-36 hours prior to anesthesia for transplantation of the allogenic transplant in the recipient.

16. The method according to claim 13, wherein the administration of cyclo-his-pro (CHP), a pharmaceutically acceptable salt thereof, stereoisomer, or a solvate thereof comprises an administering after transplantation an effective mount of the cyclo-his-pro (CHP), a pharmaceutically acceptable salt thereof, stereoisomer, or a solvate thereof as a maintenance therapy.

17. The method according to claim 13, wherein the administration of cyclo-his-pro (CHP), a pharmaceutically acceptable salt thereof, stereoisomer, or a solvate thereof and the administration of the immunosuppressant are carried out simultaneously, concurrently, or sequentially.

18. The method according to claim 13, wherein the allogenic transplant is a tissue, an organ, or cells of lung, liver, kidney, pancreas, heart, intestine, abdominal wall, scalp, uterus, or penile.

19. The method according to claim 13, wherein the effective amount of the CHP, a pharmaceutically acceptable salt thereof, stereoisomer, or a solvate thereof is about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg.
